(19) United States
(12) Patent Application Publication
(10) Pub. No.: US 2010/0106020 A1
(43) Pub. Date: Apr. 29, 2010

(54) Ultrasound System And Method Providing Wide Image Mode

(76) Inventors: Soo-Hwan Shin, Seoul (KR); Chul-An Kim, Yongin-si (KR); Sung-In Park, Hanam-si (KR)

Correspondence Address: JONES DAY, 222 EAST 41ST ST, NEW YORK, NY, 10017, US

(21) Appl. No.: 12/411,307

(22) Filed: Mar. 25, 2009

(30) Foreign Application Priority Data
Oct. 28, 2008 (KR) 10-2008-0105930

(51) Int. Cl. A61B 8/13; G06K 9/00

(52) U.S. Cl. 600/440; 382/131

(57) ABSTRACT

An ultrasound system and a method of controlling the ultrasound system are provided. The ultrasound system may receive, from a user, a selection command for a first mode among a plurality of display modes, set a target region based on region setting information that is input from the user, when the first mode is a wide mode, and extract an ultrasound image in the target region when an image extraction command is input from the user.

### CROSS-REFERENCE TO RELATED APPLICATION

This application claims the benefit of Korean Patent Application No. 10-2008-0105930, filed on Oct. 28, 2008, in the Korean Intellectual Property Office, the entire contents of which are incorporated herein by reference.

### BACKGROUND

1. Field of the Invention

Embodiments of the present invention relate to an ultrasound system and a method of controlling the ultrasound system, and more particular, to an ultrasound system providing a wide mode and a method of controlling the ultrasound system.

2. Description of the Related Art

An ultrasound system denotes a system that may emit ultrasound signals from the body surface of a subject to a selected interior portion of the body and provide images associated with blood flow or a section of soft tissue using information associated with reflected ultrasound signals. The ultrasound system is generally small and inexpensive, and also provides a display in real time. In addition, the ultrasound system has no absorbed dose such as with X rays and the like, and thus is highly stable. The ultrasound system is being widely used together with other image diagnostic apparatuses such as an X-ray diagnostic apparatus, a computerized tomography (CT) scanner, a magnetic resonance image (MRI) apparatus, a nuclear medicine diagnostic apparatus, and the like. In particular, the ultrasound system may display an interior body image in real time and thus is being variously used.

In order to prevent damage to the image quality in an output, a storage, a transmission, and the like, the existing ultrasound system may expose an ultrasound image within a limited region of a screen. For example, the ultrasound system may expose the ultrasound image within the limited region of 1024*768. However, when exposing the ultrasound image in the limited region, a portion of an image that is obtained using a probe with a relatively greater viewing angle may be omitted. Also, embodiment of a dual mode and a multi-modality may be limited. Accordingly, there is a need for an ultrasound system that may display an ultrasound image using the entire wide region of a screen.

### SUMMARY

According to an aspect of the present invention, there is provided a method of controlling an ultrasound system, the method including: receiving, from a user, a selection command for a first mode among a plurality of display modes; setting a target region based on region setting information that is input from the user, when the first mode is a wide mode; and extracting an ultrasound image in the target region when an image extraction command is input from the user.

In this instance, the image extraction may be any one of an image output, an image storage, and an image transmission.

Also, the extracting may include: exposing outline information of the target region when the image extraction command is input from the user; and extracting the ultrasound image in the target region according to exposing of the outline information.

Also, the extracting may include extracting the ultrasound image in the target region that is reset based on changed region setting information, when the changed region setting information is input from the user after exposing the outline information.

Also, the plurality of display modes may include a general mode and the wide mode.

Also, the selection command may be input by selecting an icon in a display screen of the ultrasound system and a button in a control panel of the ultrasound system.

Also, the region setting information may be set to any one of a general setting mode, a fixed ratio mode, and a fixed size mode.

According to another aspect of the present invention, there is provided an ultrasound system including: an input unit to receive, from a user, a selection command for a first mode among a plurality of display modes; a setting unit to set a target region based on region setting information that is input from the user, when the first mode is a wide mode; and an extraction unit to extract an ultrasound image in the target region when an image extraction command is input from the user.

According to embodiments of the present invention, there may be provided an ultrasound system that may select any one of a general mode and a wide mode to thereby readily control exposing of an ultrasound image and to satisfy diversified user needs, and a method of controlling the ultrasound system.

Also, according to embodiments of the present invention, there may be provided an ultrasound system that may set a target region based on region setting information, input from a user, to thereby more elastically use an ultrasound image and to improve a user convenience, and a method of controlling the ultrasound system.

Also, according to embodiments of the present invention, there may be provided an ultrasound system that may expose, for a user, outline information associated with a target region in correspondence to an output command, a transmission command, or a storage command of an ultrasound image to thereby provide the user with appropriate feedback information and to improve a degree of user freedom.

### BRIEF DESCRIPTION OF THE DRAWINGS

These and/or other aspects, features, and advantages of the invention will become apparent and more readily appreciated from the following description of exemplary embodiments, taken in conjunction with the accompanying drawings of which:

FIG. 1 is a block diagram illustrating an ultrasound system according to an embodiment of the present invention;

FIG. 2 is a flowchart illustrating a method of controlling an ultrasound system according to an embodiment of the present invention;

FIG. 3 illustrates examples of inputting region setting information and exposing outline information according to an embodiment of the present invention; and

FIG. 4 is a flowchart illustrating a method of controlling an ultrasound system according to another embodiment of the present invention.

### DETAILED DESCRIPTION

Reference will now be made in detail to exemplary embodiments of the present invention, examples of which are illustrated in the accompanying drawings, wherein like reference numerals refer to the like elements throughout. Exemplary embodiments are described below to explain the present invention by referring to the figures.

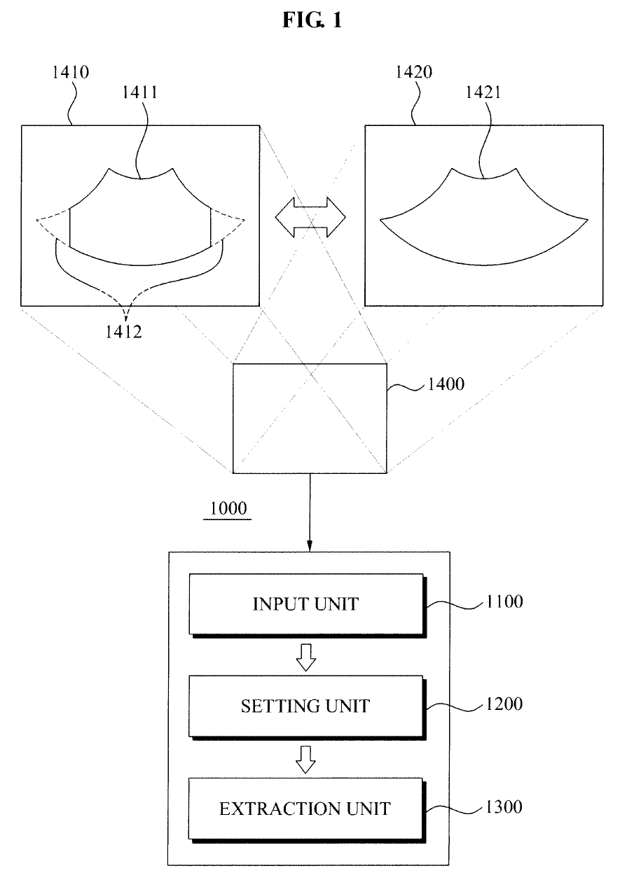

FIG. 1 is a block diagram illustrating an ultrasound system 1000 according to an embodiment of the present invention.

The ultrasound system 1000 may display an ultrasound image using a display device 1400 that is internally or externally connected to the ultrasound system 1000. The ultrasound system 1000 may display the ultrasound image via any one of a general mode screen 1410 and a wide mode screen 1420. Also, the ultrasound system 1000 may be convertible to any one between the general mode screen 1410 and the wide mode screen 1420. A general mode denotes a mode that provides an obtained ultrasound image in a limited region. For example, in the general mode, it may provide the obtained ultrasound image within a resolution region of 1024*768. As shown in FIG. 1, when displaying an obtained ultrasound image 1411 on the general mode screen 1410, left and right portions 1412 of the ultrasound image may be occluded.

A wide mode denotes a mode that provides the entire obtained ultrasound image without the limited region. For example, in the wide mode, it may provide the entire obtained ultrasound image via a screen of aspect ratio greater than or equal to 4:3. As shown in FIG. 1, when displaying an obtained ultrasound image 1421 on the wide mode screen 1420, the entire ultrasound image may be displayed. Also, the ultrasound image may be obtained via a probe connected to the ultrasound system 1000.

Referring to FIG. 1, the ultrasound system 1000 may include an input unit 1100, a setting unit 1200, and an extraction unit 1300. The input unit 1100 may receive, from a user, a selection command for a first mode among a plurality of display modes. When the first mode is a wide mode, the setting unit 1200 may set a target region based on region setting information that is input from the user. The extraction unit 1300 may extract an ultrasound image in the target region when an image extraction command is input from the user.

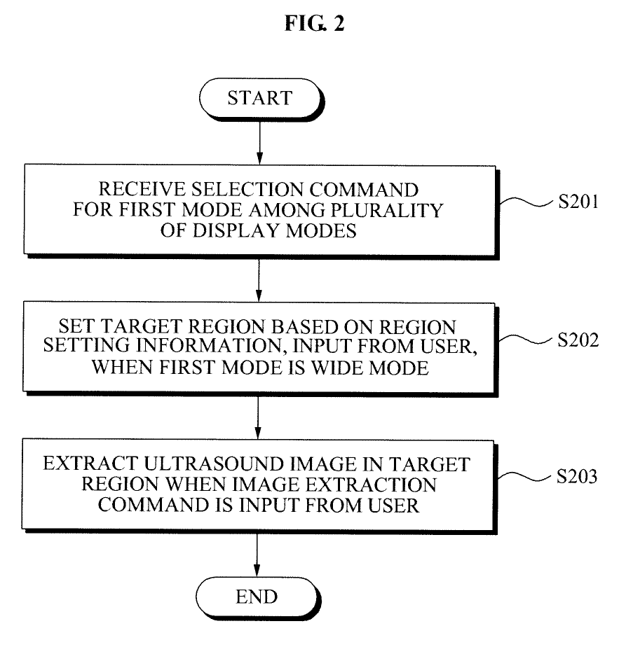

A method of controlling the ultrasound system 1000 constructed as above will be further described in detail with reference to FIGS. 2 through 4.

FIG. 2 is a flowchart illustrating a method of controlling an ultrasound system according to an embodiment of the present invention.

As shown in FIG. 2, the method of controlling the ultrasound system may be performed via operations S201 through S203. Operation S201 may be performed by the input unit 1100 of FIG. 1. Operation S202 may be performed by the setting unit 1200. Operation S203 may be performed by the extraction unit 1300.

In operation S201, the input unit 1100 may receive, from a user, a selection command for a first mode among a plurality of display modes. The plurality of display modes may include a general mode and a wide mode. Specifically, the input unit 1100 may receive the selection command for any one of the general mode and the wide mode. Also, the selection command may denote a switching command from the general mode to the wide mode, or from the wide mode to the general mode.

According to an embodiment of the present invention, the selection command may be input by selecting an icon in a particular region of a display screen of the ultrasound system 1000. Also, the selection command may be input by selecting a popup menu, a soft flexible menu, a setup dialog, or a touch panel in a display screen of the ultrasound system 1000. Also, the selection command may be input by selecting a button in a control panel of the ultrasound system 1000.

In operation S202, when the first mode is the wide mode, the setting unit 1200 may set a target region based on region setting information that is input from the user. Specifically, when the wide mode is selected from the plurality of display modes, the setting unit 1200 my receive the region setting information from the user and set the target region based on the region setting information. Here, the target region may be a portion of the entire region of an ultrasound image that is exposed in the wide mode. Specifically, the target region may be the same as the entire region of the exposed ultrasound image, or may be a partial region of the entire region.

Also, the region setting information may be input from the user for setting of the target region. Here, an input mode of the region setting information may be any one of a general setting mode, a fixed ratio mode, and a fixed size mode.

In the general setting mode, the width and the length of the target region may be arbitrarily set by the user.

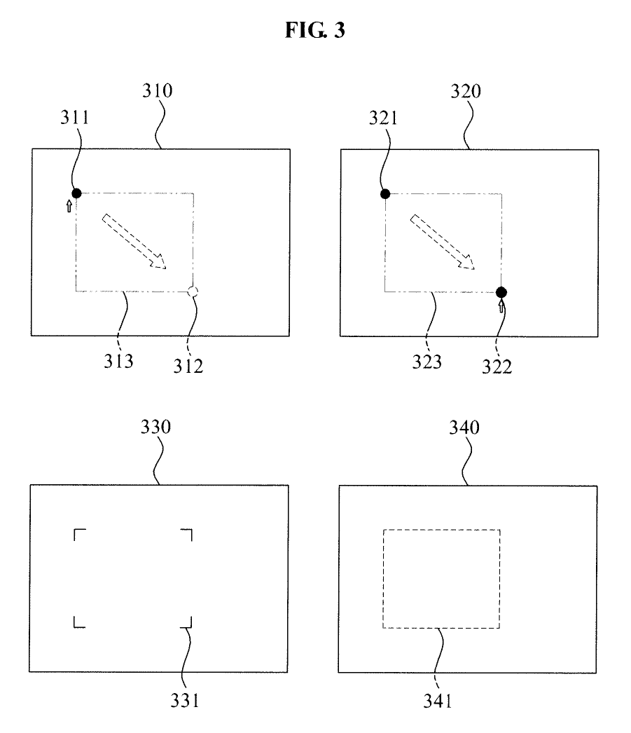

FIG. 3 illustrates examples of inputting region setting information and exposing outline information according to an embodiment of the present invention. When describing an example of inputting the region setting information according to the general setting mode with reference to FIG. 3, a user may determine a desired target region 313 by pointing a start point 311 using, for example, a mouse pointer, and dragging the mouse pointer to an end point 312 on the entire region 310. Also, the user may determine a target region 323 by pointing a first point 321 and a second point 322 on the entire region 320 using an interface.

In the fixed ratio mode, the target region may be set based on a predetermined ratio of the width to the length and points designated by the user. For example, when the ratio of the width to the length is 4:3 and the width length is input from the user using a pointer designation, the target region with 4:3 may be determined based on the input width length.

In the fixed size mode, the target region may be set based on a predetermined size of the target region and the points designated by the user. For example, when the points are input from the user, the setting unit 1200 may determine the target region with the predetermined size based on the input points.

The user may readily select any one of the general setting mode, the fixed ratio mode, and the fixed size mode. The setting unit 1200 may receive, from the user, the region setting information by any one selection of the general setting mode, the fixed ratio mode, and the fixed size mode. Also, a menu for the selection may be displayed on the display screen of the ultrasound system 1000.

Also, the region setting information may be input by selecting an icon in a particular region of the display screen of the ultrasound system 1000 or a button in a control panel of the ultrasound system 1000.

In operation S203, when an image extraction command is input from the user, the extraction unit 1300 may extract an ultrasound image in the target region. Here, the image extraction may be any one of an image output, an image storage, and an image transmission.

Specifically, when any one of an image output command, an image storage command, and an image transmission command is input from the user, the extraction unit 1300 may output, store, or transmit an ultrasound image in the target region among the entire region of the display screen of the ultrasound system 1000. The image output command, the image storage command, and the image transmission command may be input by selecting an icon in a particular region of the display screen of the ultrasound system 1000, or a button in a control panel of the ultrasound system 1000.

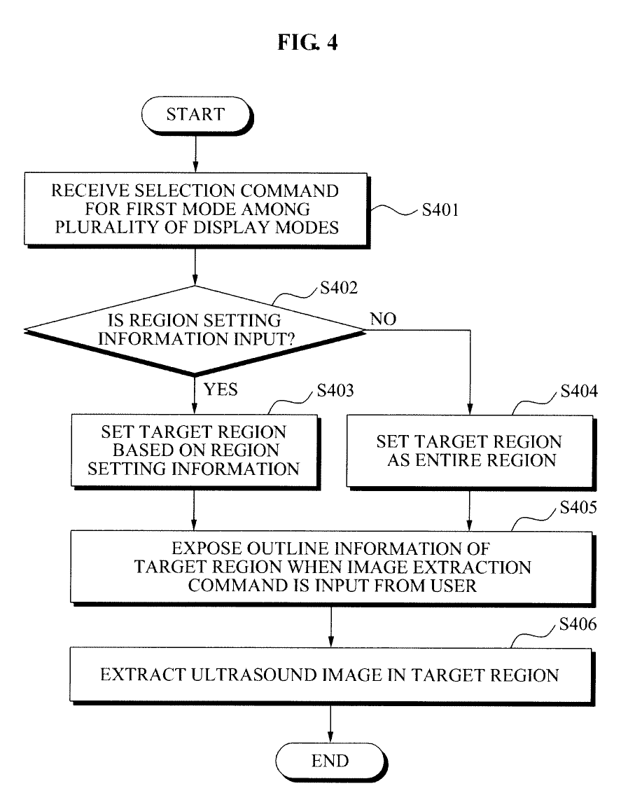

FIG. 4 is a flowchart illustrating a method of controlling an ultrasound system according to another embodiment of the present invention.

As shown in FIG. 4, the method of controlling the ultrasound system may be performed via operations S401 through S406. Operation S401 may be performed by the input unit 1100. Operations S402 through S404 may be performed by the setting unit 1200. Operations S405 and S406 may be performed by the extraction unit 1300.

In operation S401, the input unit 1100 may receive, from a user, a selection command for a first mode among a plurality of display modes. Descriptions related to operation S401 will be the same as the aforementioned descriptions related to operation S201, or may be readily induced by those skilled in the art from the descriptions. Therefore, further detailed descriptions will be omitted here.

In operation S402, the setting unit 1200 may determine whether region setting information is input from the user. When the region setting information is input, the setting unit 1200 may set a target region based on the region setting information in operation S403. The set target region may be a portion of the entire region of an ultrasound image that is exposed in a wide mode. Specifically, the target region may be the same as the entire region of the exposed ultrasound image, or may be a partial region of the entire region. Also, the region setting information may be input according to any one of a general setting mode, a fixed ratio mode, and a fixed size mode.

Conversely, when the region setting information is not input from the user in operation S402, the setting unit 1200 may set, as the target region, the entire region of the ultrasound image that is exposed in the wide mode in operation S404.

In operation S405, when an image extraction command is input from the user, the extraction unit 1300 may expose outline information of the target region. Specifically, the extraction unit 1300 may expose, for the user, the outline information of the target region in response to any one of the image extraction command, an image storage command, and an image transmission command input from the user. Through this, the user may receive feedback information regarding how the target region is set before the image output, the image storage, or the image transmission is executed.

As shown in a region 330 of FIG. 3, outline information 331 may be displayed in the form of short broken lines around four corners of the target region. Also, as shown in a region 340 of FIG. 3, outline information 341 may be displayed in a dotted square line or a solid square line indicating an outline of the target region.

In operation S406, the extraction unit 1300 may extract an ultrasound image in the target region according to displaying of the outline information. Here, the image extraction may be any one of the image output, the image storage, and the image transmission.

According to an embodiment of the present invention, when changed region setting information is input from the user after the outline information is exposed, the extraction unit 1300 may extract the ultrasound image in the target region that is reset according to the changed region setting information. Here, the changed region setting information may be input by any one of a general setting mode, a fixed ratio mode, and a fixed size mode, which is the same as the region setting information. Also, the changed region setting information may be input by selecting an icon in a particular region of a display screen of the ultrasound system 1000 or a button in a control panel of the ultrasound system 1000.

Descriptions not made above with reference to operations S401 through S406 will be the same as the descriptions made above with reference FIGS. 1 through 3, or may be readily induced from those skilled in the art from the descriptions. Therefore, further detailed descriptions will be omitted here.

The ultrasound system controlling method according to the above-described exemplary embodiments of the present invention may be recorded in computer-readable media including program instructions to implement various operations embodied by a computer. The media may also include, alone or in combination with the program instructions, data files, data structures, and the like. Examples of computer-readable media include magnetic media such as hard disks, floppy disks, and magnetic tape; optical media such as CD ROM disks and DVDs; magneto-optical media such as floptical disks; and hardware devices that are specially configured to store and perform program instructions, such as read-only memory (ROM), random access memory (RAM), flash memory, and the like. Examples of program instructions include both machine code, such as produced by a compiler, and files containing higher level code that may be executed by the computer using an interpreter. The described hardware devices may be configured to act as one or more software modules in order to perform the operations of the above-described exemplary embodiments of the present invention, or vice versa.

Although a few exemplary embodiments of the present invention have been shown and described, the present invention is not limited to the described exemplary embodiments. Instead, it would be appreciated by those skilled in the art that changes may be made to these exemplary embodiments without departing from the principles and spirit of the invention, the scope of which is defined by the claims and their equivalents.

What is claimed is:

1. A method of controlling an ultrasound system, the method comprising:
receiving, from a user, a selection command for a first mode among a plurality of display modes;
setting a target region based on region setting information that is input from the user, when the first mode is a wide mode; and
extracting an ultrasound image in the target region when an image extraction command is input from the user.

2. The method of claim 1, wherein the image extraction is any one of an image output, an image storage, and an image transmission.

3. The method of claim 1, wherein the extracting comprises:
exposing outline information of the target region when the image extraction command is input from the user; and
extracting the ultrasound image in the target region according to exposing of the outline information.

4. The method of claim 3, wherein the extracting comprises extracting the ultrasound image in the target region that is reset based on changed region setting information, when the changed region setting information is input from the user after exposing the outline information.

5. The method of claim 1, wherein the plurality of display modes includes a general mode and the wide mode.

6. The method of claim 1, wherein the selection command is input by selecting any one of an icon, a popup menu, a soft flexible menu, a setup dialog, and a touch panel in a display screen of the ultrasound system, and a button in a control panel of the ultrasound system.

7. The method of claim 1, further comprising:
setting the target region as the entire region of the wide mode when the region setting information is not input from the user.

8. The method of claim 1, wherein the region setting information is set to any one of a general setting mode, a fixed ratio mode, and a fixed size mode.

9. The method of claim 8, wherein the general setting mode receives, from the user, a designation of a start point in a display screen of the ultrasound system and receives drag information from the designated start point to an end point.

10. The method of claim 8, wherein the general setting mode receives, from the user, a first point and a second point in the display screen of the ultrasound system.

11. A computer-readable recording medium storing a program for implementing the method of claim 1.

12. An ultrasound system comprising:
an input unit to receive, from a user, a selection command for a first mode among a plurality of display modes;
a setting unit to set a target region based on region setting information that is input from the user, when the first mode is a wide mode; and
an extraction unit to extract an ultrasound image in the target region when an image extraction command is input from the user.

13. The ultrasound system of claim 12, wherein the image extraction is any one of an image output, an image storage, and an image transmission.

14. The ultrasound system of claim 12, wherein, when the image extraction command is input from the user, the extraction unit exposes outline information of the target region, and extracts the ultrasound image in the target region according to exposing of the outline information.

15. The ultrasound system of claim 12, wherein the plurality of display modes includes a general mode and the wide mode.

16. The ultrasound system of claim 12, wherein the region setting information is set to any one of a general setting mode, a fixed ratio mode, and a fixed size mode.
